(19) United States
(12) Patent Application Publication
(10) Pub. No.: US 2002/0068891 A1
(43) Pub. Date: Jun. 6, 2002

(54) Ready made charcoal compress kit

(76) Inventor: Larry A. Weathers, Talent, OR (US)

Correspondence Address: Richard C. Litman, LITMAN LAW OFFICES, LTD., P.O. Box 15035, Arlington, VA, 22215, US

(21) Appl. No.: 09/983,061

(22) Filed: Oct. 23, 2001

Related U.S. Application Data
(60) Provisional application No. 60/251,428, filed on Dec. 6, 2000.

(51) Int. Cl. A61F 13/00; A61F 15/00

(52) U.S. Cl. 602/48; 604/304; 604/307

(57) ABSTRACT

A kit having pre-made activated charcoal and gauze poultices or compresses, provided in at least three sizes, and designed for specific applications to the human body. One poultice is designed for oral use and placed between the cheek and gum. It is a linen/gauze pouch enclosing a layer of powdered charcoal (about ⅓ teaspoon). Another poultice is about 2″×2″ for a treating a spot, such as a sting or snake-bite and has a layer of powdered charcoal between linen/gauze layers. A third poultice is about 3″×5″ in size for wounds.

The pre-made activated charcoal poultices is combined with an air permeable adhesive tape to provide pre-made bandages similar to BAND-AID® bandages having the charcoal poultice substituted for the non-charcoal dressing. A kit may contain a variety of bandages sizes and may, also, include examples of the poultices, themselves.

### CROSS-REFERENCE TO RELATED APPLICATION

This application claims the benefit of U.S. Provisional Patent Application Ser. No. 60/251,428, filed Dec. 6, 2000.

### BACKGROUND OF THE INVENTION

1. Field of the Invention

The present invention relates generally to compresses. More specifically, the invention is a kit having a variety of ready-made poultices or compresses, with each having an enclosed layer of activated charcoal.

2. Description of Related Art

It is known to create a poultice or compress from charcoal, particularly in the state of activated charcoal wrapped in gauze cloth for compress treatment of sores, both in the mouth and on the exterior of the human body. It is, however, a difficult and messy job to hand-make the poultice, particularly by the person who needs it immediately. Making the poultice or compress of the correct size and so that it will not spill or leak can be a difficult and messy process.

U.S. Pat. No. 3,860,704, issued Jan. 14, 1975 to Gansen, Jr. describes a compounded product having charcoal as an ingredient for use in the mouth, but specifically excludes a gauze covering. This application of charcoal is useful only as a filler for a tooth dry-socket, and would not be appropriate for application to mouth sores.

U.S. Pat. No. 4,663,780, issued May 12, 1987, to Rawlings et al. describes a charcoal-containing pad for application to clothing. The pad consists of two layers of cotton gauze between which there is sandwiched a layer of charcoal felt. The pad necessarily is of the appropriate size for use in the armpit area of the clothing. There is no use intended for application to the human body. The pads of the '780 Patent are not sized for use as a compress or poultice for application to the human body.

U.S. Pat. No. 4,715,857, issued Dec. 29, 1987, to Juhasz et al., describes a wound dressing having a layer of charcoal cloth or felt surrounded with a permeable layer of fabric or film for contacting a wound, a semipermeable adhesive material on the opposite side, and a permeable layer bonded to the outer side of the semipermeable adhesive material. This configuration is useful for external applications, but would not be useful within the mouth, where there are sores or wounds both on the gum and the inner cheek. It also requires a special carbonized cloth and an adhesive material which may interfere with access to air, i.e., breathing of an external wound or sore. There is no provision for providing the poultice in a pre-made bandage form.

U.S. Pat. No. 4,205,685, issued Jun. 3, 1980, to Yoshida et al. describes a charcoal-containing poultice, but the charcoal is mixed with other compounds to provide heat when exposed to air. This poultice is not useful in treating wounds or open sores nor is it described as a ready-made poultice or bandage.

U.S. Pat. No. 2,960,415, issued Sep. 28, 1954, to Shuler describes a charcoal-containing blanket or dressing having a sheet of open weave material to which activated carbon granules are adhesively applied, and at least one gauze layer on each side. This material is not described as a ready-made poultice or bandage.

U.S. Pat. No. 2,066,946, issued Jan. 5, 1937, to Reiman describes a ready-made deodorizing and sterilizing bandage having in one or more embodiments the employment of activated charcoal. The charcoal is held beneath several layers of gauze and cotton or other soft absorbent fiber and is useful only as a deodorizing agent. This bandage would not be suitable or effective for direct treatment of wounds as a poultice with a single layer of gauze.

Canadian Patent No. 1,329,768, published Oct. 4, 1988, to Wright, et al. describes a wound dressing having a carbonized and activated cellulosic layered between two facing layers of woven or non-woven fabric. There is no mention of providing particular sizes of ready-made poultices, or making them part of a ready-made bandage.

Canadian Patent No. 1,212,879, published Oct. 21, 1986, to Laszlo describes a wound dressing having a carbonized cloth layer between a semipermeable layer and a biodegradable layer. There is no mention of providing particular sizes of ready-made poultices, or making them part of a ready-made bandage.

None of the above inventions and patents, taken either singly or in combination, is seen to describe the instant invention as claimed.

### SUMMARY OF THE INVENTION

The present invention solves the problem of assembling charcoal and gauze poultices or compresses by providing at least three sizes of pre-made poultices or compresses specifically designed for specific applications to the human body in a kit. One poultice is for oral use and, more particularly for placement between cheek and gum and is about 1″×½″ in size and consists of a linen/gauze pouch enclosing a layer of activated powdered charcoal(about ⅓ teaspoon). Another size is for a spot such as a sting or snake-bite and is about 2″×2″ in size and contains a layer of powdered activated charcoal between linen/gauze layers. A third is about 3″×5″ in size for sores, wounds, etc. and contains a layer of powdered activated charcoal between linen/gauze layers. These poultices are simple and easily made, and have a long shelf life, making them appropriate for placement in a first-aid kit, or medicine cabinet, for example.

Another aspect of the invention is the use of the pre-made activated charcoal poultices in combination with an air permeable adhesive tape to provide pre-made bandages similar to BAND-AID® bandages having the activated charcoal poultice substituted for the non-charcoal dressing. Various sizes of the pre-made bandages may be provided, preferably corresponding to existing sizes of the BAND-AID® bandages. The pre-made charcoal poultice bandages are covered on the adhesive side with a release strip and packaged for sterility and cleanliness. The poultice bandages may be pre-wetted and packed in foil as previously described for the poultice, itself. A kit may contain a variety of bandage sizes and may, also, include examples of the poultices, themselves. Accordingly, it is a principal object of the invention to provide a kit having a variety of sizes of pre-made charcoal and gauze poultices or compresses.

It is another object of the invention to provide a kit having as one of its components, a charcoal and gauze poultice which is sized for use in the mouth of a human being.

It is a further object of the invention to provide a kit having as one component a charcoal poultice for application to an external spot injury to a human being such as a snake-bite or a sting.

Still another object of the invention is to provide a kit having as one component a charcoal and gauze poultice for use as a compress for sores and external wounds.

Yet another object of the invention is to provide a ready-made bandage having an activated poultice incorporated into the bandage.

Still, another object of the invention is to provide a kit having a variety of sizes of pre-made bandages having an activated charcoal poultice incorporated into the bandage.

It is an object of the invention to provide improved elements and arrangements thereof in a kit for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is a perspective view of a ready-made poultice, sized for oral application according to the present inventive kit.

FIG. 2 is a sectional view taken along lines 2-2 of the oral application-sized poultice of FIG. 1.

FIG. 3 is a plan view of a ready-made poultice, sized for application to a spot injury according to the present inventive kit.

FIG. 4 is a plan view of a ready-made poultice as in FIG. 3 depicted in a partially broken away foil wrapper.

FIG. 5 is a plan view of a ready-made poultice, sized for compress use for external wounds or sores according to the present inventive kit.

FIG. 6 is an environmental perspective view of the ready-made poultice of FIG. 5, applied as a compress according to the present inventive kit.

FIG. 7 is a plan view of a kit according to the present invention having a kit box without the box top, and showing various poultices placed inside according to the present invention.

FIG. 8A is a plan view of a charcoal poultice as above incorporated in a small spot ready-made bandage.

FIG. 8B is a plan view of a charcoal poultice as above incorporated in a large spot ready-made bandage.

FIG. 8C is a plan view of a charcoal poultice as above incorporated in a small rectangular ready-made bandage.

FIG. 8D is a plan view of a charcoal poultice as above incorporated in a large rectangular ready-made bandage.

FIG. 8E is a plan view of a charcoal poultice as above incorporated in a small medical size rectangular ready-made bandage.

FIG. 8F is a plan view of a charcoal poultice as above incorporated in a large medical size rectangular ready-made bandage.

### DETAILED DESCRIPTION OF THE PREFERRED EMBODIMENTS

The present invention solves the problem of assembling charcoal and gauze poultices or compresses by providing a kit having at least three sizes of pre-made poultices or compresses specifically designed for specific applications to the human body.

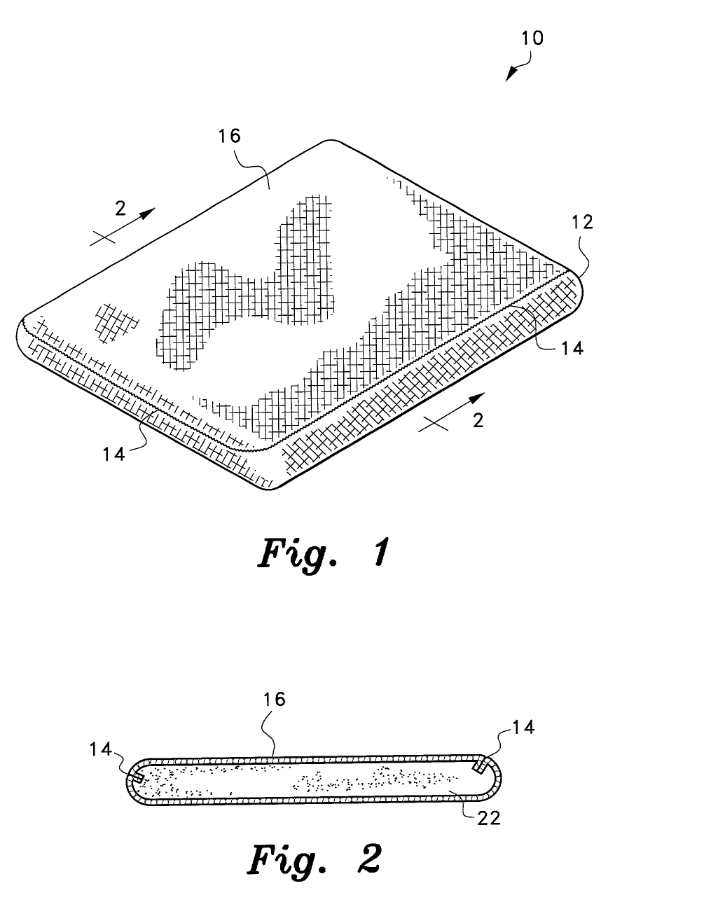

Referring to FIGS. 1 and 2, there is shown a view in perspective of a poultice sized for oral use, and, particularly for placement between the cheek and gum and is about 1″×½″ in size, wherein mouth applied poultice 10 comprises linen/gauze pouch 12 enclosing a layer 22 of powdered activated charcoal (about ⅓ teaspoon). During assembly, a linen/gauze pouch 16 is sewn into a pouch configuration as see seam 14, Before seam closure, pouch 12 is filled with the appropriate amount of charcoal 22.

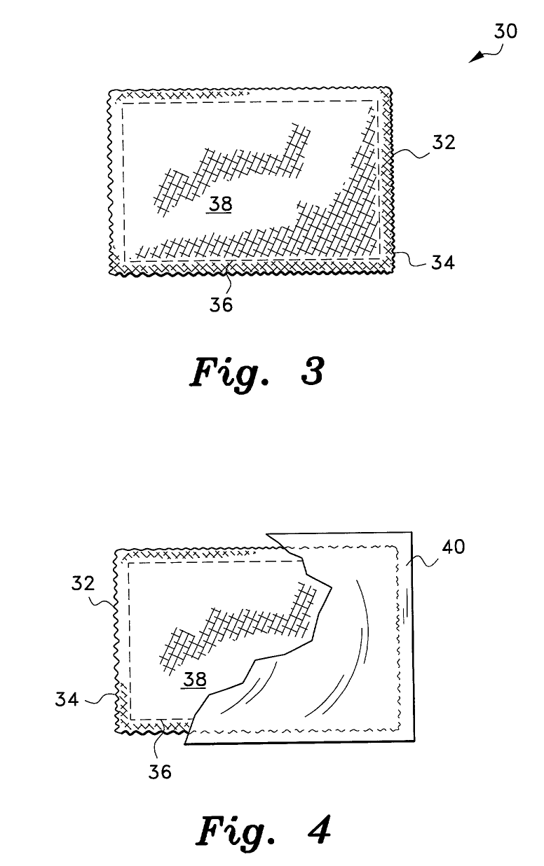

Referring to FIG. 3, there is shown a plan view of another size poultice 32 of spot wound poultice system 30, such as a sting or snake-bite and is about 2″×2″ in size and contains a layer of powdered activated charcoal between linen/gauze layers. It is constructed by taking two pieces 34 of linen/gauze of the appropriate size and dusting one side of each piece. The two pieces are placed together so that the charcoal-dusted sides face each other, forming a charcoal layer. The gauze pieces are then attached together around their perimeter by any convenient means such as by adhesives or sewing as see seam 36 to form charcoal poultice portion 38.

Referring to FIG. 4, there is shown spot wound poultice 32 within a partially torn away foil wrapper 40.

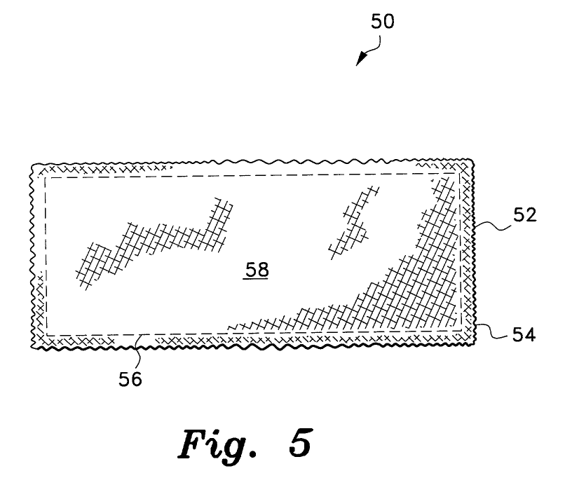

Referring to FIG. 5, there is shown a plan view of a third size poultice 52 of poultice system 50, useful as a compress and is about 3″×5″ in size so as to be useful as a compress for external sores or wounds, etc. and is made in a similar manner as the 2″×2″ size, above, with two pieces of linen/gauze 54 having charcoal dusted sides facing each other and sown to form seam 56, thus, forming charcoal poultice portion 58.

Of course, poultices of dimensions differing from those described above may be provided in the inventive kit as desired.

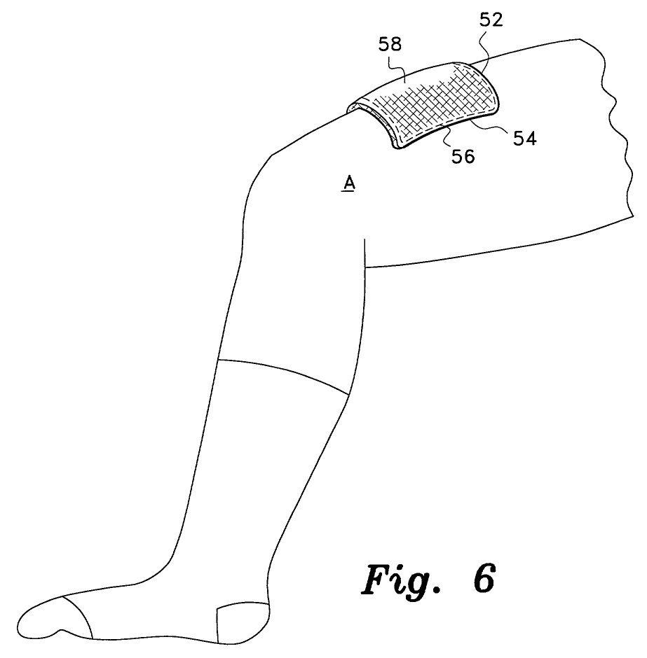

Referring to FIG. 6, there is shown an environmental perspective view of the wound compress poultice of FIG. 5 applied to a human thigh, A.

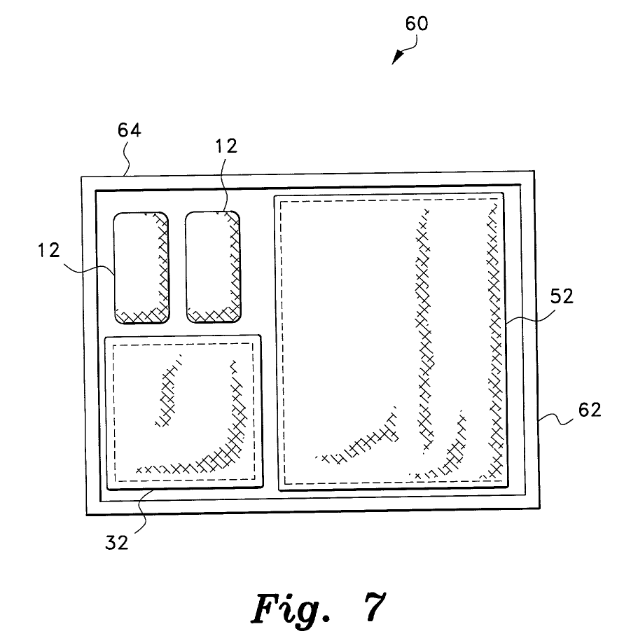

Referring to FIG. 7, there is shown a plan view of a kit according to the present invention with the lid (not shown) removed, and wherein kit 62 of charcoal poultice kit system 60 comprises a kit container 64, the enclosed kit containing a variety of the above-described poultices, e.g., mouth-applied poultices 12, spot wound poultices 32, and external sore or wound compress poultices 52. The compress poultice 52 may be folded over in the kit in the interest of compactness. The contents of the kit may contain any number and size of poultices as desired. The kit may include a housing of any desired container type, such as a metal or plastic enclosed box, and may be part of a larger first-aid kit.

Although it is contemplated that powdered charcoal, preferably activated charcoal, is employed in the assembly of the various described poultices, solid charcoal wafers, granulated charcoal, carbonized cloth, or compressed charcoal briquettes may be substituted in some applications as found desirable.

The inventive poultices within the kit intended for external body use are preferably wetted with olive oil, mineral oil, or water and the like, to stimulate its effectiveness in soaking up liquid material emanating from a wound. The individual poultices may be individually wrapped in conformable, liquid impermeable coverings such as those made of plastic film or metal foil wrap and can be accessed by tearing the covering to expose the poultice desired. The pre-wrapped poultice may be wrapped in a dry condition and wetted upon use, or the pre-wrapped poultice may be wetted and then wrapped so as to be ready for immediate use. This embodiment would be particularly desirable for immediate use where olive oil or mineral oil is not immediately available. The mouth-employed poultice need not be pre-wetted, since saliva will act as a wetting agent.

The charcoal poultices of the present inventive kit are simple, inexpensive, and have a long shelf-life, such that the kit may be incorporated in a larger kit such as a general first-aid kit, or conveniently located in a medicine cabinet, for example.

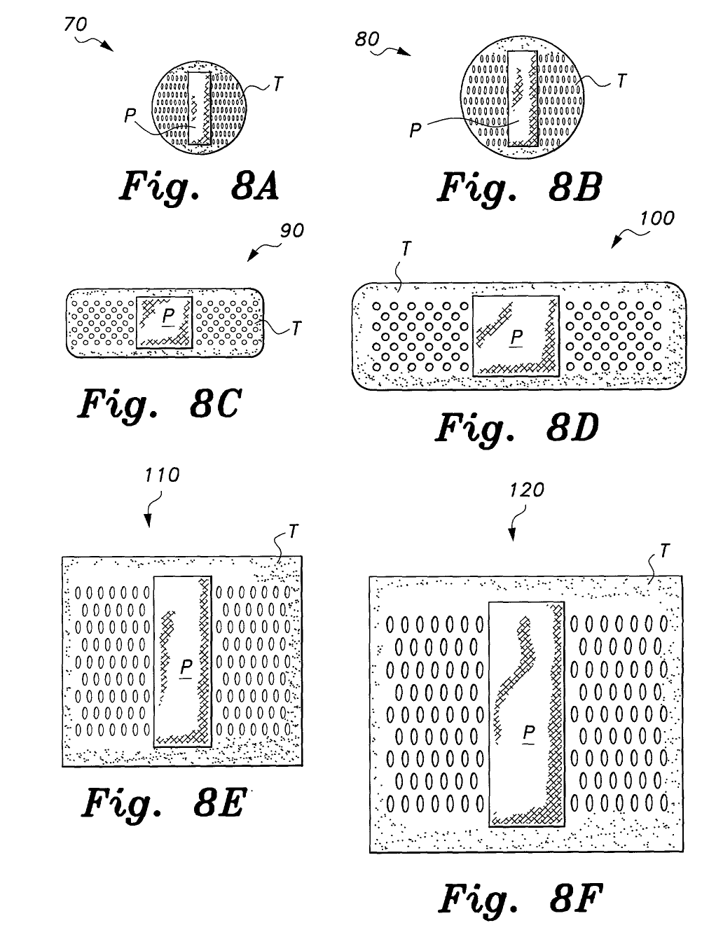

Referring to FIGS. 8A-8F, the charcoal poultices of the present invention may be incorporated in a pre-made bandage. The appropriate size of poultice T is centrally attached to an adhesive tape-like material T which is preferably air-permeable. The ready-made bandage may be provided with a release liner which may be peeled off the adhesive side of the tape-like material and uncover the poultice in a known manner. This embodiment is similar to that of a BAND-AID® adhesive bandage with the inventive poultice substituted for the carbon-free dressing. The poultices P and the adhesive material or tape T may correspond in size with standard BAND-AID® sizes, including 1″ round 70, 2″ round 80, standard strip-type small-⅝″×2¼″ 90 and large-¾″×3″ 100, and extra large-3″×6″ 110 and medical sizes of 4″ square and larger 120 as seen in FIGS. 8A-8F, respectively. The poultice size is selected as appropriate for the particular bandage but should be of such size relative to the adhesive material as to provide sufficient coverage for the wound while leaving adequate surface for maintaining adherence to the skin. For example, the standard small bandage may have a poultice of about 3/8″ square and the standard large bandage may have a poultice of about ¼″ square. It is noted that the poultice bandage is preferably configured such that the adhesive tape T completely surrounds the poultice P so as to completely seal it against the skin. The preferred poultice configuration is that of FIG. 3, above, sized appropriately for the individual bandage. As above, such as shown in FIG. 4, the poultice bandage may be packaged dry or pre-wetted and packaged in liquid impermeable foil or similar wrapping to maintain sterility and cleanliness. If the poultice bandage is dry packaged, the poultice portion must be wetted as by water, mineral oil, or olive oil before application to the wound as described above. The poultice bandage is then placed over the wound and the adhesive tape-like material pressed on the surrounding skin to form the applied bandage. A kit may be made to include any or all of the poultices and poultice bandages as desired and appears similar to that of FIG. 7. The kit may also include the oral poultice of FIGS. 1 and 2 since a bandage is not required in this application.

It is to be understood that the present invention is not limited to the embodiments described above, but encompasses any and all embodiments within the scope of the following claims.

I claim:

1. A pre-made poultice kit having at least three constituent members of varying planar dimensions, each of said members comprising a poultice made up of at least one layer of charcoal located between two layers of gauze or linen and closed around its perimeter.

2. The poultice kit according to claim 1, having at least one sealed bag comprising said two layers of gauze or linen having a perimeter of about 1 inch by 1/2 inch and containing about ⅓ of a teaspoon of powdered activated charcoal for oral use and, more particularly, between the cheek and gum.

3. The poultice kit according to claim 2, wherein said two layers of gauze or linen are sewn in a pouch configuration having an inner seam.

4. The poultice kit according to claim 1, further comprising a plurality of spot wound compresses having two layers of gauze having a perimeter of about 2 inches by 2 inches and containing at least one layer of activated charcoal between said layers of gauze.

5. The poultice kit according to claim 4, wherein said two layers of gauze, containing said layer of charcoal, is closed by attachment of said layers of gauze around their perimeter by adhesive or sewing.

6. The poultice kit according to claim 5, wherein said spot-wound compress is wrapped in a foil wrapper.

7. The poultice kit according to claim 1, wherein said two layers of gauze comprise a large wound compress having a perimeter of about 3 inches by 5 inches and contains a layer of activated charcoal between said layers of gauze.

8. The poultice kit according to claim 7, wherein said two layers of gauze, containing said layer of charcoal, is closed by attachment of said layers of gauze around said perimeter by adhesive or sewing.

9. The poultice kit according to claim 8, wherein said kit contains a plurality of said large wound compresses in folded form.

10. The poultice kit according to claim 1, wherein said at least three constituent members are enclosed in a box made of plastic or other appropriate material.

11. A pre-made activated charcoal poultice bandage comprising a poultice made up of at least one layer of charcoal located between two layers of gauze or linen and closed around its perimeter, said poultice being attached on one of said layers of linen or gauze to a web having one side covered with adhesive such as adhesive tape, said poultice being generally centrally mounted on said adhesive-covered side of said web;
whereby said pre-made bandage is placed such that the poultice covers a wound and said adhesive-covered web is attached to the users skin so as to retain said poultice in contact with the wound.

12. The pre-made activated charcoal poultice bandage of claim 11, said web being perforated so as to be permeable to air so as to allow air to and gases to pass through to and away from said poultice when worn by the user.

13. The pre-made activated charcoal poultice bandage of claim 11, wherein said bandage is wrapped to maintain sterility and cleanliness.

14. The pre-made activated charcoal poultice bandage of claim 11, wherein said bandage is pre-wetted and wrapped in foil or plastic to maintain dampness in said poultice.

15. The pre-made activated charcoal poultice bandage of claim 11, wherein said bandage is a spot bandage of about 1 inch to about 2 inches in overall diameter and having a said charcoal poultice centrally mounted along a diameter of said bandage.

16. The pre-made activated charcoal poultice bandage of claim 11, wherein said bandage is a strip type bandage of from about ⅝″×2¼″ to about ¾×3″ and having said charcoal poultice centrally mounted along said strip.

17. The pre-made activated charcoal poultice bandage of claim 11, wherein said bandage is a rectangular type bandage for medical uses of 4″ and over in overall size and having an appropriate sized charcoal poultice centrally mounted thereon.

18. A kit comprising a variety of sizes of the pre-made activated charcoal poultice bandages of claim 11.

19. The kit of claim 18, further comprising pre-made activated charcoal poultices in addition to said charcoal poultice bandages.

20. The kit of claim 18, wherein said pre-made activated charcoal poultices comprise at least one sealed bag comprising two layers of gauze or linen having a perimeter of about 1 inch by inch and containing about ⅓ of a teaspoon of powdered charcoal for oral use and, more particularly, between the cheek and gum.
